(19) United States
(12) Patent Application Publication
(10) Pub. No.: US 2026/0076655 A1
(43) Pub. Date: Mar. 19, 2026

(54) BIOPSY DEVICES AND METHODS

(71) Applicant: COOK MEDICAL TECHNOLOGIES LLC, BLOOMINGTON, IN (US)

(72) Inventors: Jeffrey L. Scifert, Arlington, TN (US); Juan Sebastián Sánchez López, Pereira (CO); Dan Velasco, Fallbrook, CA (US); Christian Tostado, Temecula, CA (US); Jeff Alan Burke, Fallbrook, CA (US)

(73) Assignee: COOK MEDICAL TECHNOLOGIES LLC, BLOOMINGTON, IN (US)

(21) Appl. No.: 19/327,070

(22) Filed: Sep. 12, 2025

Related U.S. Application Data
(60) Provisional application No. 63/694,439, filed on Sep. 13, 2024.

(51) Int. Cl. A61B 10/02; A61B 90/00

(52) U.S. Cl. CPC A61B 10/0275; A61B 2010/0208; A61B 2090/3954

(57) ABSTRACT

The present disclosure provides medical devices and methods for using said medical devices. In particular, the present disclosure provides biopsy devices wherein the protrusion distance of the inner stylet can be selected and an integrated depth stop member is configured to allow the device to align with a single length guide cannula in accordance with the selected protrusion distance.

### BACKGROUND

The present invention relates generally to medical technology and in particular aspects to methods and systems for tissue biopsy. Biopsy procedures are critical in the diagnosis and treatment of various medical conditions, including cancers and other diseases characterized by abnormal tissue growth. A biopsy involves the removal of a small tissue sample from the body for further examination. To obtain an accurate diagnosis, it is essential to collect tissue samples from the precise location of interest while minimizing damage to surrounding healthy tissues.

Traditional biopsy devices often consist of a biopsy needle comprising a cannula and a stylet. The stylet is often configured to hold a portion of patient tissue as the cannula is fired over the stylet. However, conventional biopsy devices have several limitations that can hinder the accuracy and safety of the procedure. Significant challenges include the inability to visualize the position of the sample notch and/or control the depth of needle penetration with sufficient precision. Over-penetration can result in damage to underlying structures, while under-penetration may lead to insufficient tissue sampling, compromising diagnostic accuracy.

Furthermore, the ability to visualize the biopsy needle during the procedure, particularly in imaging-guided biopsies, is crucial. Magnetic resonance imaging (MRI)-guided biopsies, for example, require that the biopsy needle and its components be visible on the MRI scan to ensure accurate targeting of the tissue of interest. However, many existing biopsy devices lack features that enhance their visibility under MRI, making it difficult for clinicians to guide the needle accurately to the target site.

Another challenge with existing biopsy devices is the lack of adaptability to different tissue depths and sizes. Biopsy procedures often require varying levels of needle penetration depending on the location and type of tissue being sampled. Prior devices required manual attachment of a separate depth stop member, a step that is cumbersome, may be forgotten, or is prone to error as the depth stop length must coincide with the desired needle penetration. Traditional devices that do not allow for automatic adjustment of needle protrusion distances may not be versatile enough to accommodate the needs of different biopsy procedures.

There remain needs for improved and/or alternative biopsy devices as well as methods for making such devices. The present disclosure is addressed to those needs.

### SUMMARY

In certain aspects, the present disclosure pertains to biopsy devices, in particular with biopsy devices configured to allow selection of sample size.

Accordingly, in one form, the present disclosure provides a biopsy device comprising a biopsy needle having a cannula and a stylet slidably received within the cannula. In some forms, the biopsy device comprises a housing having a distal end, wherein the biopsy needle extends from the distal end of the housing. The housing comprising a firing mechanism configured to extend the stylet relative to the cannula such that a distal end of the stylet protrudes from a distal end of the biopsy needle cannula by a selectable protrusion distance. The firing mechanism may also be configured to fire the cannula relative to the stylet such upon firing the distal end of the cannula moves in a distal direction towards the distal end of the stylet. In certain embodiments, the selectable protrusion distance is adjustable between at least a first distance and a second longer distance. The biopsy device may also include a depth stop member positioned at the distal end of the housing and coaxially positioned around a portion of the biopsy needle. In some forms, the depth stop member is configured to extend from the housing when the first protrusion distance is selected, and to retract at least partially into the housing when the second protrusion distance is selected. In certain embodiments, the stylet comprises a sampling notch positioned near the distal end of the stylet. In some forms, the device comprises one or more MRI visible markers positioned proximal and/or distal to the sampling notch. In some forms, the depth stop member has a depth stop extended length between the distal end of the housing and a contact surface on the depth stop member configured to contact a guide cannula hub, and wherein the depth stop extended length is about the difference between the first protrusion distance and the second protrusion distance. In accordance with some forms, the first protrusion distance is about 10 mm. In accordance with some forms, the second protrusion distance is about 20 mm.

In some forms the present disclosure provides a biopsy kit comprising a biopsy needle as described above and a coaxial guide needle sized to receive the biopsy needle. In certain embodiments, the distal end of the housing is configured to contact a proximal end of the coaxial guide needle, such that a distal end of the coaxial guide needle aligns with a distal end of the biopsy needle when the first protrusion distance is selected. In certain embodiments, the depth stop member is configured to contact a proximal end of the coaxial guide needle when the first protrusion distance is selected, such that the distal end of the biopsy needle aligns with the distal end of the coaxial guide needle.

In certain embodiments, the present disclosure provides methods of obtaining a tissue same using the devices described herein. In some forms, such methods may comprise inserting the biopsy needle through a guide cannula, the distal end of the guide cannula positioned near a target tissue, wherein the depth stop member contacts a hub body of the guide cannula when the distal end of the biopsy needle is positioned at the distal end of the guide cannula. In accordance with some forms, such methods may comprise retracting the depth stop member into the housing.

Still further embodiments, as well as features and advantages of embodiments described herein, will be apparent to persons skilled in the relevant field from the descriptions herein.

### BRIEF DESCRIPTION OF THE DRAWINGS

FIG. 1 provides a perspective view of one embodiment of a biopsy device of the present disclosure.

FIG. 2A provides a side view of one embodiment of a coaxial guide needle of the present disclosure.

FIG. 2B provides a side view of one embodiment of a coaxial guide needle separated into a needle portion and a guide cannula of the present disclosure.

FIG. 3A provides a side view of one embodiment of a biopsy device of the present disclosure.

FIG. 3B provides a side view of one embodiment of a biopsy device of the present disclosure.

FIG. 4A provides a side view of one embodiment of a biopsy device of the present disclosure.

FIG. 4B provides a side view of one embodiment of a biopsy device of the present disclosure.

FIG. 5 provides a side view of one embodiment of a puller component of a biopsy device of the present disclosure.

FIG. 6 provides a side view of one embodiment of a depth stop mechanism component of a biopsy device of the present disclosure.

FIGS. 7A-B provide opposing side views of one embodiment of a slider component of a biopsy device of the present disclosure.

FIG. 8 provides a cutaway side view of one embodiment of a biopsy device of the present disclosure.

FIGS. 9A-B provides cutaway side views of one embodiment of a biopsy device of the present disclosure.

FIGS. 10A-B provides cutaway side views of one embodiment of a biopsy device of the present disclosure.

FIG. 11 provides a cutaway side view of one embodiment of a biopsy device of the present disclosure.

FIG. 12A is a side view of one embodiment of a use of the device of the present disclosure.

FIG. 12B is a side view of one embodiment of a use of the device of the present disclosure.

FIG. 12C is a side view of one embodiment of a use of the device of the present disclosure.

FIG. 12D is a side view of one embodiment of a use of the device of the present disclosure.

FIG. 13 is a partial side view of one embodiment of a device of the present disclosure.

FIG. 14 is a top front right-side perspective view of one embodiment of a device of the present disclosure.

FIG. 15 is a bottom rear left-side perspective view of one embodiment of a device of the present disclosure.

FIG. 16 is a front elevation view of one embodiment of a device of the present disclosure.

FIG. 17 is a rear elevation view of one embodiment of a device of the present disclosure.

FIG. 18 is a left-side elevation view of one embodiment of a device of the present disclosure.

FIG. 19 is a right-side elevation view of one embodiment of a device of the present disclosure.

FIG. 20 is a top down view of one embodiment of a device of the present disclosure.

FIG. 21 is a bottom up view of one embodiment of a device of the present disclosure.

### DETAILED DESCRIPTION

Reference will now be made to certain embodiments, some of which are illustrated in the drawings, and specific language will be used to describe the same. It will nevertheless be understood that no limitation of the scope of the disclosure is thereby intended. Any alterations and further modifications in the described embodiments and any further applications of the principles as described herein are contemplated as would normally occur to one skilled in the art to which this disclosure relates.

“Distal,” as used herein, refers to a location or portion of the device that is positioned farther from the operator or user during normal operation.

“Proximal,” as used herein, refers to a location or portion of the device that is positioned closer to the operator or user during normal operation.

As disclosed above, aspects of the present disclosure relate to biopsy devices. In particular, aspects of the present disclosure relate to biopsy devices designed for the precise collection of tissue samples. Such biopsy devices may be particularly useful for procedures where precise control over tissue sampling depth is critical, such as in breast, liver, or prostate biopsies. As discussed further herein, the devices and methods of the present disclosure enable accurate needle placement and tissue sample collection while avoiding damage to surrounding tissues.

In certain embodiments, the present disclosure provides a biopsy device comprising a biopsy needle. In some forms, the biopsy needle comprises a cannula, and a stylet that is slidably received within the cannula. The biopsy needle cannula serves as an outer tube through which the stylet extends. In accordance with some forms, the cannula may be formed from any suitable biocompatible material, for example: inconel, titanium, stainless steel, or other suitable alloy. In some forms, the biopsy needle cannula comprises one or more depth marks along its length, configured to provide visual feedback relating to the depth of insertion of the distal end of the biopsy needle. The distal end of the cannula may include a cutting edge. For example, in some forms the cannula's distal end is sharpened to form a cutting edge that facilitates penetration of the biopsy needle into patient tissues, and/or facilitates separation of tissue samples during device firing.

In accordance with various embodiments of the present disclosure, the biopsy device may incorporate either an automatic or semi-automatic firing mechanism to facilitate precise tissue sampling. In the automatic firing configuration, a single activation of the device's firing mechanism causes the stylet to extend rapidly, followed immediately by the firing of the cannula, ensuring swift and accurate capture of the tissue sample. This configuration may be particularly advantageous in scenarios requiring minimal tissue displacement and rapid sample acquisition, for example when sampling a calcified tumor. Conversely, the device may also feature a semi-automatic firing mechanism, wherein the stylet is initially advanced to a first position by partially depressing the plunger, allowing for precise placement, visualization (e.g. MRI, etc.) and assessment before tissue capture. Upon further pressing the plunger, the cannula is then fired, enveloping the tissue portion within the sampling notch. This semi-automatic approach provides the user with greater control over the biopsy process, allowing for a more deliberate and adjustable procedure.

Devices of the present disclosure may also include a stylet slidably received within the biopsy needle cannula. In some forms, the stylet is a solid rod that can be extended and/or retracted relative to the biopsy needle cannula to acquire a tissue sample. In accordance with some forms, the stylet comprises a sampling notch positioned near the distal end of the stylet. The sampling notch may comprise an indentation of the stylet configured to capture a patient tissue sample. In some forms, the stylet comprises one or more visible markers positioned proximal and/or distal to the sampling notch. Such markers allow for real-time imaging and precise positioning of the needle during medical imaging procedures, such as MRI-guided biopsy procedures.

In accordance with the present disclosure, biopsy devices may be enhanced through the incorporation of various markers, including MRI visible markers and markers visible to the naked eye. These markers can be strategically positioned on the stylet and/or cannula to facilitate precise real-time imaging during MRI-guided biopsy procedures. Potential MRI visible markers may include materials or coatings that exhibit high contrast under MRI scanning, such as gadolinium-based compounds, iron oxide particles, or other MRI-visible materials. Additionally, chemical etching techniques may be employed to create visible patterns or grooves directly on the surface of the stylet or cannula. These etched markers can serve as alignment guides, ensuring accurate positioning of the biopsy needle in relation to the target tissue. In some embodiments, the markers may be located proximal to the sampling notch on the stylet to provide a clear visualization of the needle's position. Additional markers may be placed distal to the sampling notch, aiding in confirming the complete insertion or retraction of the stylet within the cannula. The integration of multiple types of visible markers significantly enhances the accuracy of needle placement and tissue sample collection, reducing the risk of damage to surrounding tissues and improving procedural outcomes.

Biopsy devices of the present disclosure include a housing portion. In some forms, the housing serves as a grip for the user to secure and manipulate the biopsy device. In accordance with some forms, the housing comprises an elongate structure extending from a proximal end to a distal end. In some forms, the biopsy needle extends from the distal end of the housing. In certain embodiments, the housing includes a firing mechanism which may be actuated by a user while holding the housing. The firing mechanism is designed to provide controlled movement of the cannula relative to the stylet. In accordance with some forms, the firing mechanism comprises a plunger positioned at or near the proximal end of the housing. In certain embodiments the firing mechanism may include a spring-loaded plunger that is operably connected to the stylet and/or cannula such that then the plunger is actuated, the stylet is extended forward causing its distal end to protrude from the distal end of the biopsy needle cannula by a predetermined distance, herein referred to as “protrusion distance”. In accordance with some forms, the device is configured to allow selection between two or more protrusion distances. In some forms, the device is configured to allow selection between a first protrusion distance and a second protrusion distance. It is within the scope of the disclosure to provide a device having more than two selectable protrusion distances. For example, a device may be provided having any number of predetermined protrusion distances configured to be used with various lengths of coaxial guide needles. In one exemplary embodiment the device is configured to allow selection between a first protrusion distance of 10 mm and a second protrusion distance of 20 mm.

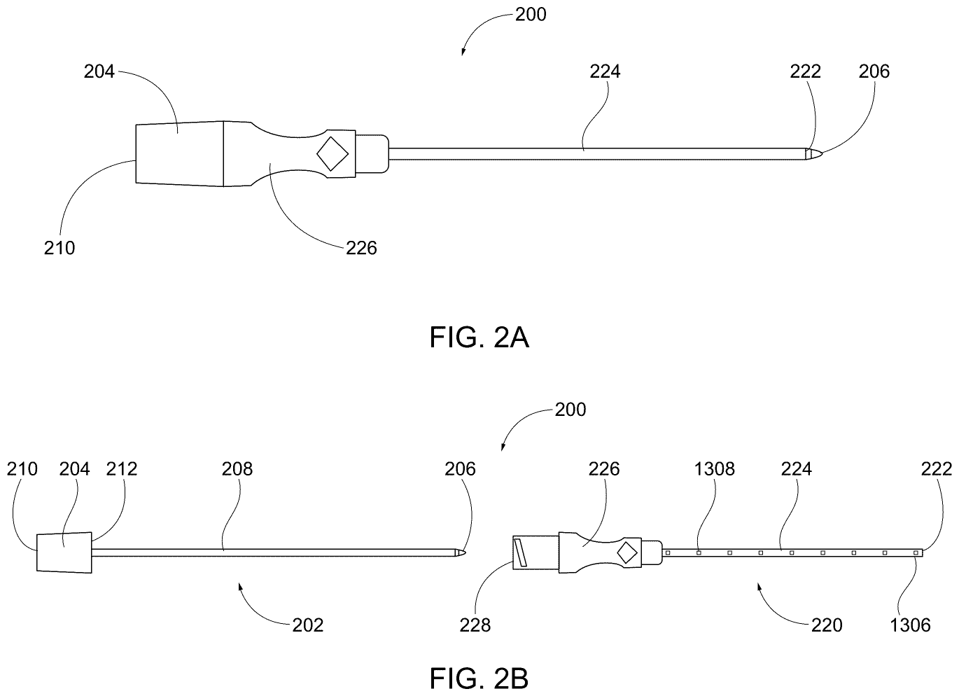

The housing may include one or more indicia to facilitate use. For example, in certain embodiments the housing comprises an indicia indicating the axial position of the notch on the biopsy needle. This alignment helps the user to easily identify the location of the sampling notch within the patient's tissue, enhancing the accuracy of the biopsy procedure. In accordance with some forms devices of the present disclosure may include indicia positioned on the guide cannula. With reference to FIG. 2B, guide cannula 220 may include one or more depth markers 1308 positioned along cannula body 224. Depth markers 1308 may be visible to the naked eye and/or imageable under medical imaging (e.g. magnetic resonance imaging, ultrasound, x-ray, etc.). In accordance with some form the depth markers 1308 are positioned at regular intervals along the length of cannula body 224. For example, depth markers 1308 may be spaced every 5 millimeters, every 10 millimeters, or every 20 millimeters. In some forms distal depth marker 1306 is positioned at the distal end of 222 of cannula body 224. Additional indicia may include one or more color coding indicia. For example, devices may be provided wherein one or more of the plunger 126, grip 132, depth stop member 150, and/or hub body 226 are colored to indicate a feature of the device such as cannula gauge, needle length, firing depth, etc.

In some forms, biopsy devices of the present disclosure feature a depth stop member. In some forms the depth stop member is configured to retract (proximally) and extend (distally) based on the selected protrusion distance. The protrusion distance may be selected based on the length of the coaxial guide needle ensuring alignment with the coaxial guide needle's length. Broadly speaking, retraction involves shortening the depth stop member in a proximal direction, allowing the distal tip of the stylet to be positioned precisely where the distal tip of the coaxial guide needle was previously located. This mechanism ensures the biopsy needle's length is effectively aligned with the guide needle's length, facilitating accurate tissue sampling.

In accordance with some forms, the depth stop member is coaxially positioned around a portion of the biopsy needle. In some forms, the depth stop member is coaxially positioned around a portion of the biopsy needle near the distal end of the housing.

When a shorter protrusion distance is selected, the depth stop member may act as a stop for the coaxial guide needle. In certain embodiments, depth stop member provides a contact surface that limits the insertion depth when the coaxial guide needle is introduced.

When a longer protrusion distance is selected, the depth stop member may retract, partially or wholly, into the housing. In such cases, the depth stop member retracts proximally, allowing the proximal end of the coaxial guide needle to contact the distal end of the housing. In certain embodiments the distal end of the depth stop member may be aligned with the distal end of the housing and provide some or all of the contact surface with the proximal end of the coaxial guide needle.

It is within the scope of the disclosure to provide a device wherein the depth stop member does not protrude from the housing and the coaxial needle guide may be inserted into the housing at varying depths to contact the depth stop member. In this way the depth stop member serves to limit the depth of needle penetration during a biopsy procedure, in particular when a relatively shorter protrusion distance is selected and insertion of the biopsy needle though the coaxial guide needle to the point of contact with the distal end of the housing would result in the biopsy needle protruding from the end of the coaxial guide needle prior to firing and risk missing the biopsy target or damaging surrounding tissue. Thus, in some forms, the depth stop member is configured to retract into the housing when a relatively longer protrusion distance is selected. In some forms such retraction allows the proximal end of the coaxial guide needle to contact the distal end of the housing. Conversely, when a relatively shorter protrusion distance is selected, the depth stop member extends distally from the housing, effectively limiting the needle's penetration depth to prevent over-insertion into the tissue.

In devices configured to allow selection between two or more protrusion distances, the length of the depth stop member may correspond to the difference between the first and second protrusion distances. This design ensures that the needle penetrates the tissue to the desired depth, with the depth stop member either in a retracted or extended position depending on the selected protrusion distance.

Devices of the present disclosure may be provided as part of a medical kit. Such components of the kit may be provided in sterilized form, for example in a sterile pouch. In some forms the disclosure provides a biopsy kit, which includes a biopsy device as disclosed herein and a coaxial guide needle sized to receive the biopsy needle. Exemplary coaxial guide needles include trocar needles. Such coaxial guide needles may comprise an elongate cannula having a hub positioned at a proximal end. In some forms, the hub is configured to engage a portion of the depth stop member. In accordance with some forms, the coaxial guide needle is MRI-visible. In some forms, the coaxial guide needle is designed such that when the biopsy needle is inserted, the distal end of the biopsy needle aligns with the distal end of the coaxial guide needle. In this way the user is able to accurately place the biopsy needle prior to firing. In some forms, when the longer protrusion distance is selected, the housing's distal end contacts the proximal end of the coaxial guide needle. In some forms, when the shorter protrusion distance is selected, the depth stop member contacts the proximal end of the coaxial guide needle, ensuring that the distal ends of both needles remain aligned.

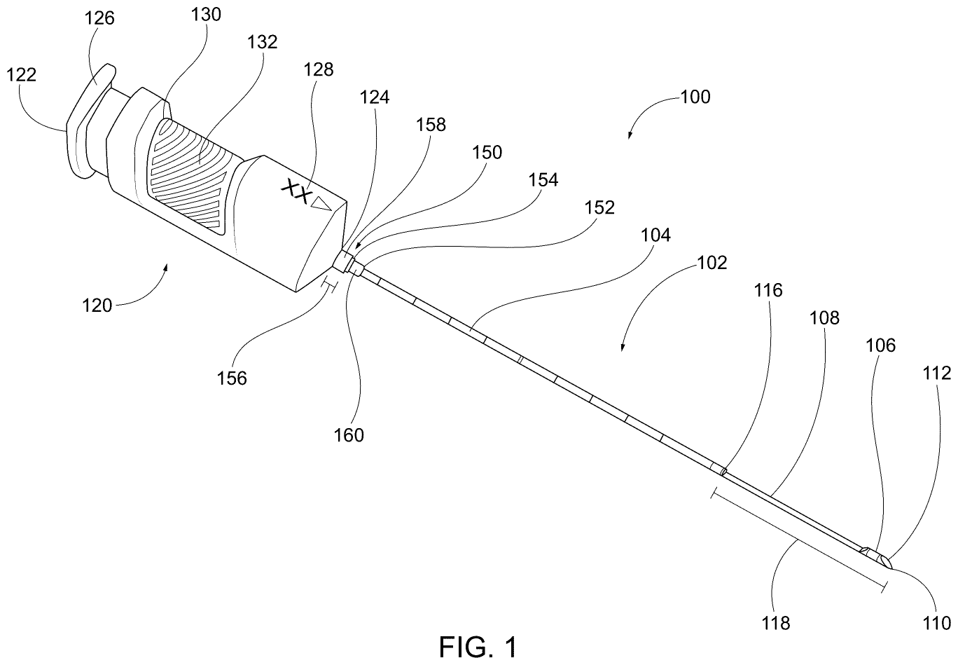

The embodiment shown in FIG. 1 includes a biopsy device 100 having biopsy needle 102 and housing 120. In the illustrated embodiment, biopsy needle 102 includes cannula 104 and stylet 106. Stylet 106 includes notch 108 configured to receive a patient tissue portion, and beveled end 112 at stylet distal tip 110. As shown in the illustrated embodiment, stylet 106 is configured to protrude from distal end 116 of cannula 104. Protrusion distance 118 comprises the length of stylet 106 extending from cannula distal end 116 to stylet distal tip 110 upon firing. In the illustrated embodiment housing 120 comprises an elongate structure extending from proximal end 122 to distal end 124. Plunger 126 is positioned at proximal end 122 to facilitate firing of stylet 106, for example by depressing plunger 126. The illustrated embodiment includes indicia 128, which as described above may indicate a variety of parameters, including for example, the axial position of notch 108 and/or the size of needle 102. Housing 120 may include a recessed portion 130 to facilitate handling and orientation of the device. In the illustrated embodiment recessed portion 130 is axially aligned with notch 108. The device may also include grip 132 also configured to facilitate handling and orientation of the device. Depth stop member 150 is shown extending from distal end 124 of housing 120. In the illustrated embodiment, depth stop member 150 is shown in the extended configuration which, as described above, is useful when the device is configured for a shorter protrusion distance. Biopsy needle 102 extends from housing distal end 124 and through depth stop member 150. Depth stop member 150 comprises proximal portion 158, shoulder 154, and distal portion 160. In the illustrated embodiment, shoulder 154 is shown as a tapered surface extending between proximal portion 158 and distal portion 160. It is within the scope of the disclosure to allow for a shoulder 154 which is not tapered, for example comprising a surface extending about 90 degrees from the surface of the proximal portion 158 and the distal portion 160. In the illustrated embodiment, proximal portion 158 is radially enlarged related to distal portion 160.

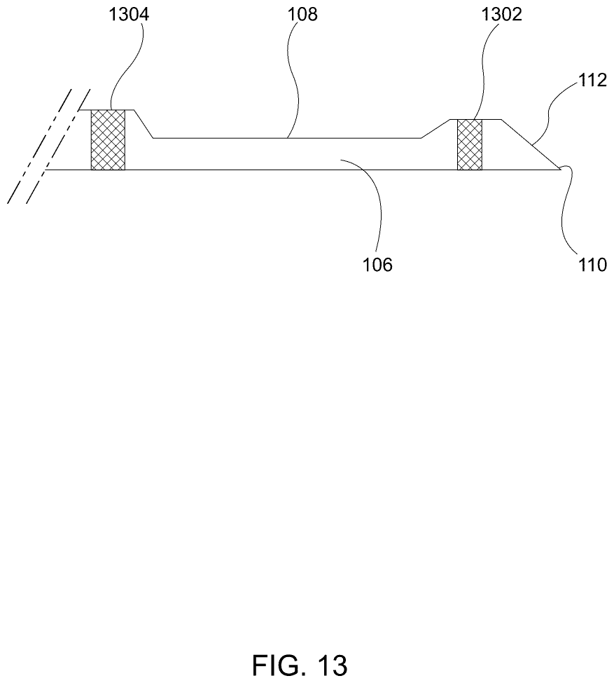

FIG. 13 illustrates a distal portion of one embodiment of stylet 106. Distal notch marker 1302 is positioned distal to notch 108. Proximal notch marker 1304 is positioned proximal to notch 108. As described above, such notch markers enable the notch position to be imageable under medical imaging (e.g. magnetic resonance imaging, ultrasound, x-ray, etc.). Notch markers 1032, 1304, and depth markers 1306, 1308 may comprise any suitable material allowing for medical imaging of the marker. In some forms, notch markers 1032, 1304, and/or depth markers 1306, 1308 are electroplated markers. In some forms, notch markers 1032, 1304, and/or depth markers 1306, 1308 are chemically etched into the stylet and/or or cannula. It is within the scope of the disclosure to provide for a device having both electroplated and chemically etched markers. For example, chemically etched markers may be configured to be visible to the naked eye while not imageable under MRI or other medical imaging modality. In this way, a cannula may be provided having alternating electroplated depth markers 1308, visible under medical imaging, and chemically etched depth markers 1308, visible to the naked eye.

FIGS. 2A and 2B illustrate one embodiment of a coaxial guide needle 200 for use with the biopsy devices of the present disclosure. Coaxial guide needle 200 includes needle portion 202 and guide cannula 220. Needle portion 202 extends from needle portion proximal end 210 to needle portion distal tip 206. Needle shaft 208 is configured such that distal tip 206 extends from guide cannula distal end 222 when needle portion is fully inserted into guide cannula 220. Needle shaft 208 protrudes from needle control member 204. Control member 204 is configured to facilitate removal of needle portion 202 from guide cannula 220. Guide cannula 220 extends from proximal end 228 to distal end 222. Guide cannula 220 includes cannula body 224 and hub body 226. Hub 206 includes proximal end 228 which is configured for contact with control member distal surface 212 when needle portion 202 is fully inserted into guide cannula 220.

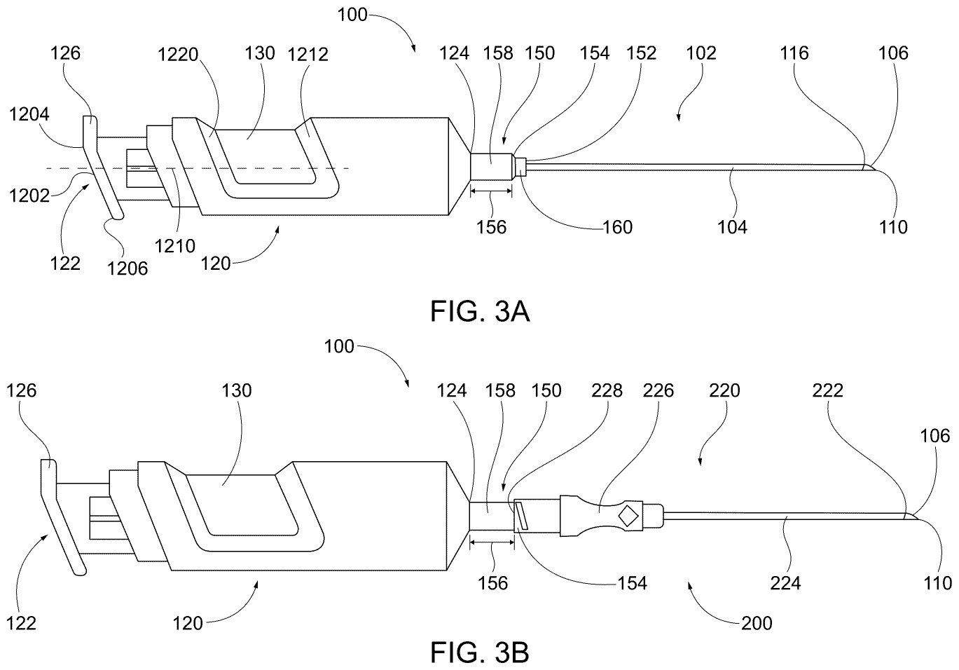

FIGS. 3A and 3B illustrate an embodiment of a biopsy device 100 and coaxial guide needle similar to those described in FIGS. 1, 2A, and 2B. In the illustrated embodiment, biopsy device 100 is loaded in a ready to fire position. Distal tip 110 of stylet 106 is positioned at distal end 116 of cannula 104. In the illustrated embodiments stylet 106 is shown as protruding from distal end 116 of cannula 104, however it is within the scope of the disclosure to provide a device where the distal tip of the stylet is aligned with the distal end of the cannula, or positioned within the cannula near the distal end prior to firing. Biopsy device 100 is shown with depth stop member 150 in the extended position. In the illustrated embodiment, depth stop member 150 has a protrusion length 156 extending from the distal end 124 of housing 120 to shoulder 154. FIG. 3B illustrates an embodiment wherein biopsy needle 102 of biopsy device 100 is positioned within guide cannula 220 of coaxial guide needle 200. As shown, depth stop member comprises a shoulder 154 that contacts the proximal end 228 of the hub body 226, such that the distal end 116 of cannula 104 is positioned at or near distal end 222 of the guide cannula 220. Hub body 226 is sized to receive distal portion 160 of depth stop member 150. In the illustrated embodiments stylet 106 is shown as protruding from distal end 222 of guide cannula 220, however it is within the scope of the disclosure to provide a device where the distal tip of the stylet is aligned with the distal end of the guide cannula, or positioned within the guide cannula near the distal end prior to firing.

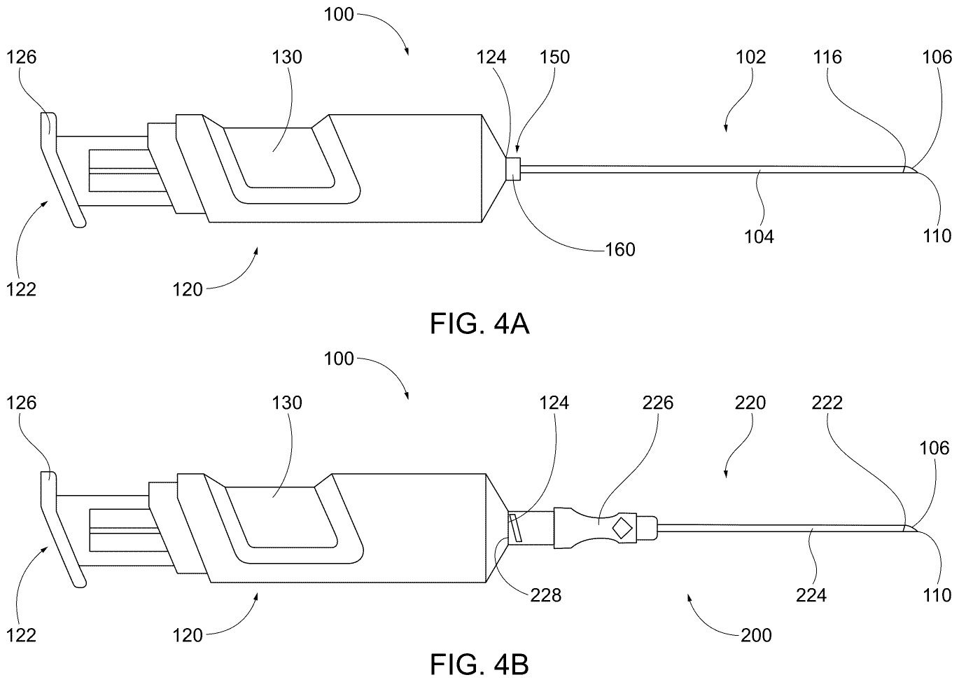

FIGS. 4A and 4B illustrate an embodiment of a biopsy device 100 and coaxial guide needle similar to those described in FIGS. 1, 2A, and 2B. In the illustrated embodiment, biopsy device 100 is loaded in a ready to fire position. Distal tip 110 of stylet 106 is positioned at distal end 116 of cannula 104. In the illustrated embodiments stylet 106 is shown as protruding from distal end 116 of cannula 104, however it is within the scope of the disclosure to provide a device where the distal tip of the stylet is aligned with the distal end of the cannula, or positioned within the cannula near the distal end prior to firing. Biopsy device 100 is shown with depth stop member 150 in the retracted position. FIG. 4B illustrates an embodiment wherein biopsy needle 102 of biopsy device 100 is positioned within guide cannula 220 of coaxial guide needle 200. As shown, the distal end 124 of the housing 120 contacts the proximal end 228 of the hub body 226 such that the distal end 116 of cannula 104 is positioned at or near distal end 222. In the illustrated embodiments stylet 106 is shown as protruding from distal end 222 of the guide cannula 220, however it is within the scope of the disclosure to provide a device where the distal tip of the stylet is aligned with the distal end of the guide cannula, or positioned within the guide cannula near the distal end prior to firing. In the illustrated embodiment, distal portion 160 of depth stop member 150 is shown extending from distal end 124 of housing 120. In such embodiments, the hub body 226 of the guide cannula 220 is sized to receive distal portion 160.

Thus, the present disclosure provides biopsy devices which can readily be switched between multiple sample sizes (protrusion distances) while utilizing a single length guide cannula. When loading a larger sample size (longer protrusion distance) the depth stop member retracts inside the housing enabling a length match between the biopsy needle and the guide cannula. When loading a shorter sample size (shorter protrusion distance), the depth stop member is extended to fill the gap distance which would otherwise be present between the biopsy device housing and the guide cannula to match the length of the biopsy needle and the guide cannula positioning the tip of the biopsy needle at the distal end of the guide cannula.

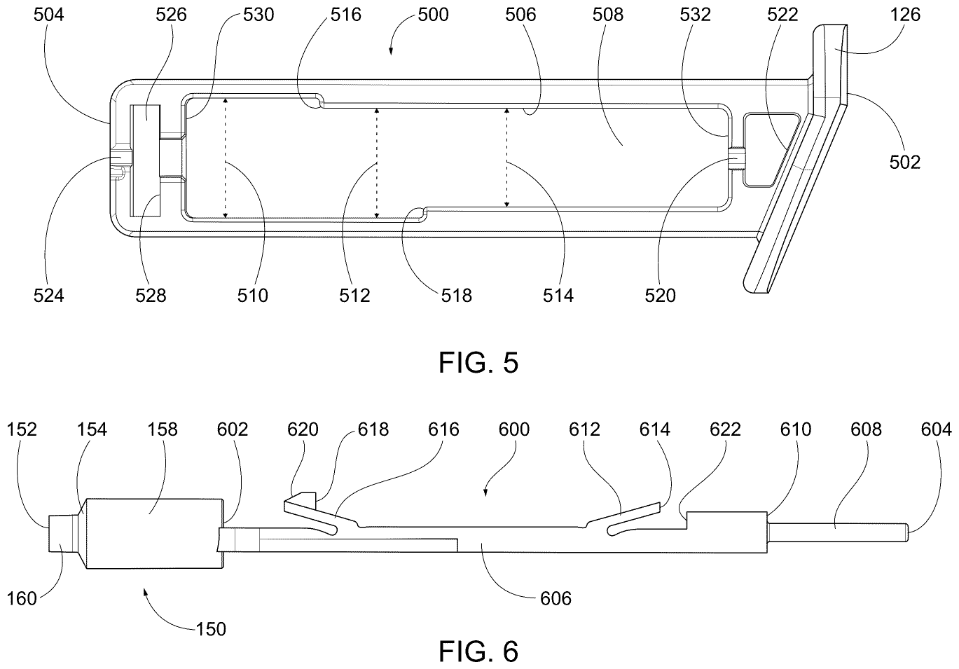

Turning now to a discussion of an exemplary embodiment of a biopsy needle device as shown in FIGS. 5-12. In accordance with some forms, a biopsy needle device 100 as disclosed herein may comprise a puller 500, a depth stop mechanism 600, and/or a slider 700. Puller 500 extends from proximal end 502 to distal end 504. Plunger 126 is positioned at or near distal end 502. Slider cavity wall 506 defines slider cavity 508 sized to receive slider 700. Slider cavity 508 having varying widths between opposing faces of slider cavity wall 506. In the illustrated embodiment, slider cavity 508 includes a distal width 510, an intermediate with 512, and a proximal width 514. In the illustrated embodiment the width decreases progressing from distal width 510, intermediate width 512, and proximal width 514. The change in width is caused at least in part by distal ridge 516 and proximal ridge 518. Puller 500 may also include one or more grooves for allowing passage of the other components of the devices as described herein. For example, proximal groove 520 is sized to allow stylet 106 to pass through to stylet attachment point 522, in this way stylet 106 moves with puller 500. Distal groove 524 is configures to allow passage of a portion of depth stop mechanism 600, and/or biopsy needle 102 including cannula 104 and stylet 106. In accordance with some forms, puller 500 includes reset cavity 526 defined by reset cavity wall 528.

In accordance with some forms, depth stop mechanism 600 comprises depth stop member 150 and control member 606. In the illustrated embodiment control member 606 is attached to depth stop member 150 at or near the proximal end 602 of depth stop member 150. Depth stop mechanism 600 extends from proximal end 604 to depth stop member distal end 152. Control member 606 may be configured to secure a spring to facilitate extension of depth stop member 150. In the illustrated embodiment, end portion 608 is shown having a diameter configured to allow end portion 608 to pass through the central lumen of a suitable spring. Spring contact surface 610 is positioned to secure the spring and to facilitate compression of the spring. Control member 606 includes one or more deflectable arms. Proximal arm 612 extends from control member 606 at angle generally towards proximal end 604, and includes proximal contact surface 614. Slider contact surface 622 is positioned proximal to proximal arm 612 and faces towards distal end 152. Distal arm 616 extends from control member 606 at an angle generally towards distal end 152 and includes distal contact surface 618, which faces towards proximal end 604, and tapered surface 620.

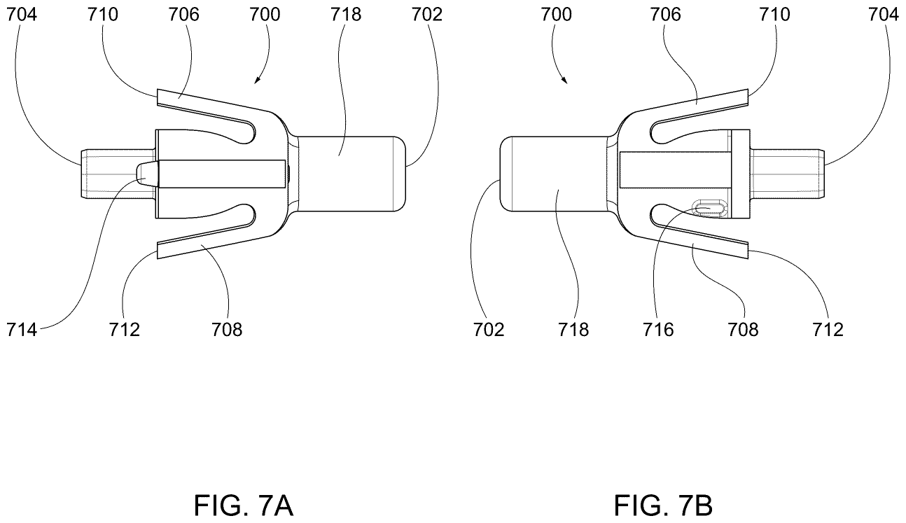

FIGS. 7A and 7B illustrate one embodiment of slider 700. Slider 700 extends from slider proximal end 702 to slider distal end 704 and comprises a first deflectable slider arm 706 and a second deflectable slider arm 708. First slider arm 706 includes first contact surface 710, facing towards distal end 704. Second slider arm 708 includes second contact surface 712, facing towards distal end 704. In accordance with some forms, slider 700 includes bump stop 714 and/or tab 716. In accordance with some forms, distal end 704 of slider 700 is configured to receive cannula 104 such that the cannula moves with the slider. In accordance with some forms, slider 700 includes a spring receiving portion 718 at or near the proximal end, the spring receiving portion configured to receive a firing spring.

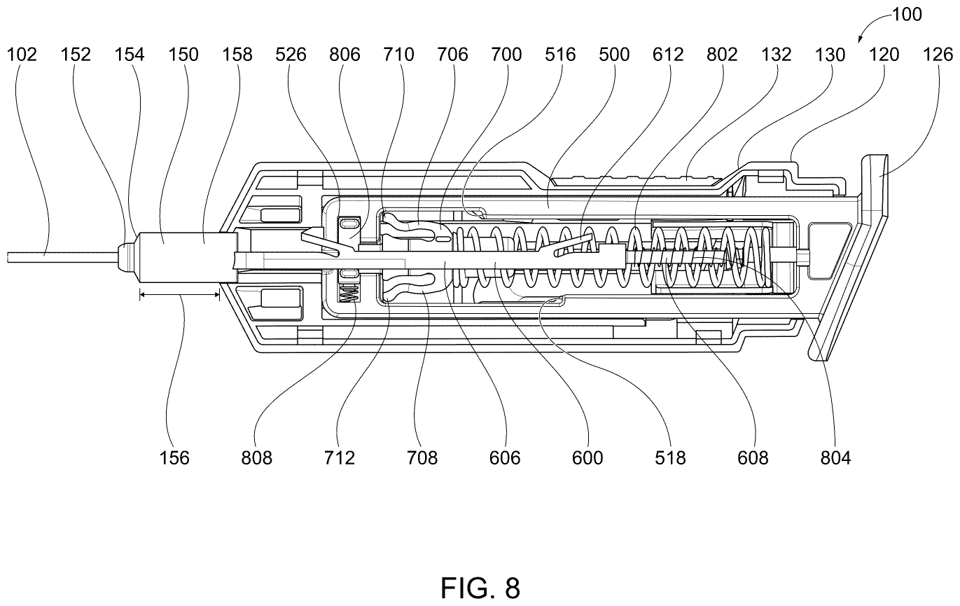

FIGS. 8-11B are partial cutaway views of one embodiment of biopsy device 100 as disclosed herein. In the illustrated embodiments the components described above (e.g. puller 500, depth stop mechanism 600, and slider 700) are positioned in housing 120 and shown during various stages of the device's use cycle. With reference to FIG. 8, the device is shown in a resting position. Firing spring 802 and depth stop spring 804 are uncompressed, or under less tension than when in a compressed state. Reset mechanism 806 and reset spring 808 are positioned in reset cavity 526.

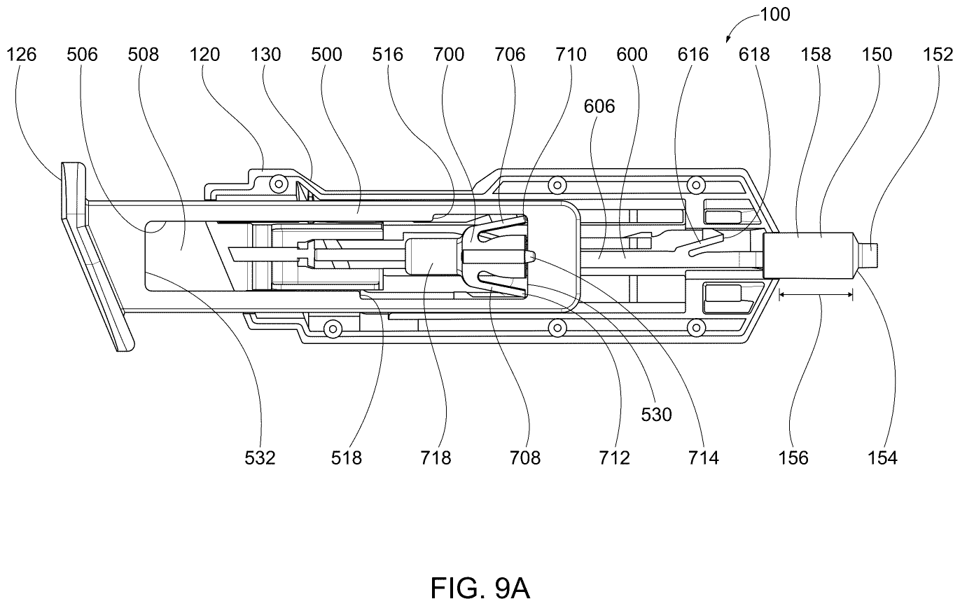

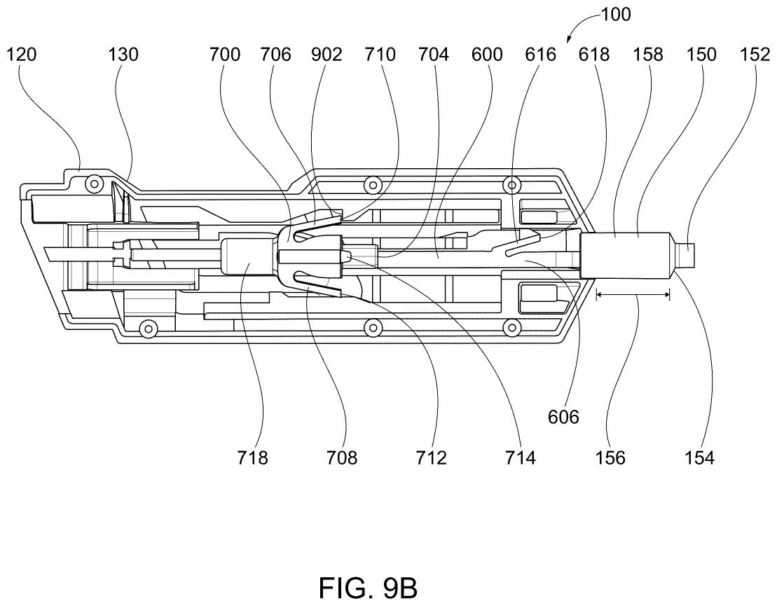

FIGS. 9A and 9B illustrate the device when the shorter sample size is selected. In some embodiments the shorter sample size comprises a 10 mm sample. Depth stop member 150 is in the extended position. In some forms, proximal arm contact surface 614 of depth stop mechanism 600 contacts a distal facing internal protrusion (not pictured) of housing 120 in order to lock depth stop member in the extended position and prevent undesired retraction of the depth stop member. Puller 500 is retracted in a proximal direction causing slider 700 to also move in a proximal direction. Slider first arm contact surface 710 is engaged by a first protrusion 902 of housing 120 thus locking the device in the ready position. Firing spring (not pictured) is compressed by retraction of puller 500. Movement of puller 500 in a distal direction, for example by pressing plunger 126, causes distal ridge 516 to deform slider first arm 706 releasing slider 700 from the lock position. The firing spring forces the slider, and the cannula (not pictured) to rapidly move in a distal direction to take the biopsy sample. In FIG. 9B the puller 500 is omitted to allow visualization of slider 700 in the locked position described.

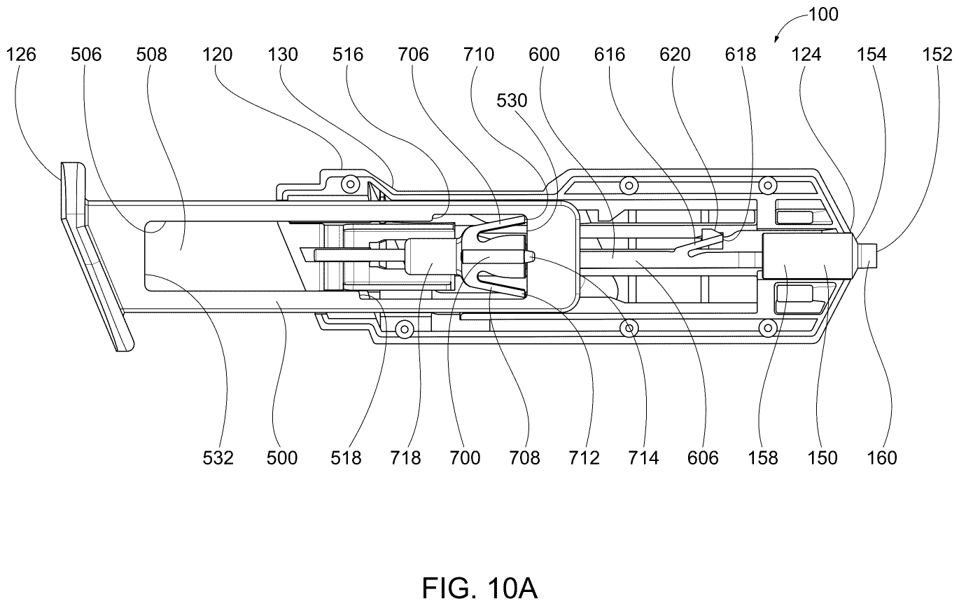

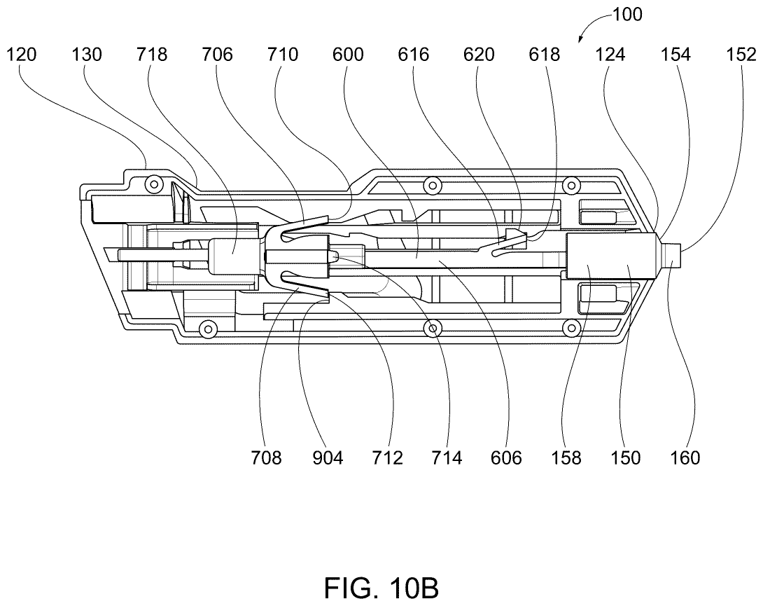

FIGS. 10A and 10B illustrate the device when the longer sample size is selected. In some embodiments the longer sample size comprises a 20 mm sample. Puller 500 is retracted in a proximal direction causing slider 700 to also move in a proximal direction. Slider second arm contact surface 712 is engaged by a second protrusion 904 of housing 120 thus locking the device in the ready position. Slider 700 causes proximal arm 612 of depth stop mechanism 600 to deform, for example by tab 716 passing over proximal arm 612. Such deformation releases the depth stop mechanism 600 from its extended position. Slider 700 causes depth stop mechanism 600 to move in a proximal direction, retracting at least the proximal portion 158 of depth stop member 150 into the housing. In some forms, tab 716 pushes against slider contact surface 620 forcing the depth stop mechanism 600 in a proximal direction. Such movement causes retraction of the depth strop member 150 and compression of the depth stop spring (not pictured). Firing spring (not pictured) is compressed by retraction of puller 500. Movement of puller 500 in a distal direction, for example by pressing plunger 126, causes proximal ridge 518 to deform slider second arm 708 releasing slider 700 from the lock position. The firing spring forces the slider, and the cannula (not pictured) to rapidly move in a distal direction to take the biopsy sample. In FIG. 10B the puller 500 is omitted to allow visualization of slider 700 in the locked position described.

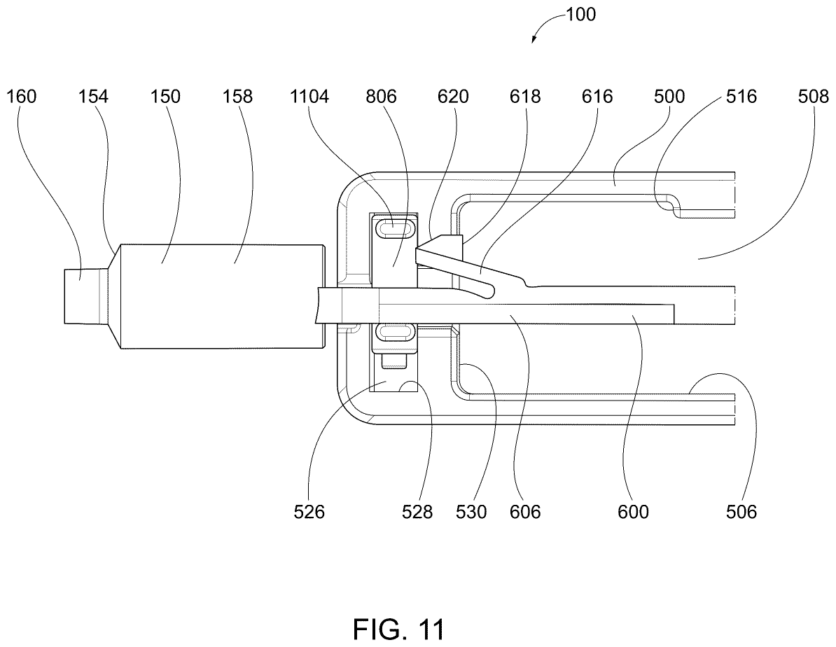

In accordance with some forms, the device includes a mechanism for returning the depth stop mechanism to the extended position. With reference to FIG. 11, shown is one embodiment of a biopsy device as disclosed. FIG. 11 shows the distal portions of slider 700 and depth stop mechanism 600. Reset tab 1102 is positioned in the reset cavity 526. Reset spring (Not shown) biases the tab in the upper position as shown. In this way when the puller 500 is moved distally, for example to take a biopsy sample, distal arm 616 of depth stop mechanism 600 is moved past reset tab projection 1104. Once the slider is released and moves to the end position the reset tab is forced down, compressing the reset tab spring (not shown). Such movement may occur, for example, when a distal portion of the slider contacts a beveled receiving area of the reset tab. With the reset tab in the down position, retraction of the puller will cause the reset tab projection 1104 to deform distal arm 616 as it moves over tapered surface 620. Such deformation releases the contact between the distal arm and the housing allowing force from the depth stop spring to push the depth stop mechanism in a distal direction and resetting the device to the resting position described above.

In use, the biopsy devices provided herein are configured to automatically extend and/or retract a depth stop member in response to a selected protrusion distance. The present disclosure includes methods of using the devices described herein to obtain a short sample and/or a long sample by selection of a short protrusion distance or a long protrusion distance. As used herein a short protrusion distance refers to a protrusion distance which is smaller than the alternative large protrusion distance. For example, in some forms the short protrusion distance comprises a protrusion distance having a length of 1 mm to 20 mm, while the large protrusion distance comprises a protrusion distance having a length greater than 20 mm. Exemplary protrusion distances which may be selected as either the short and/or long protrusion distance include but are not limited to: 5 mm, 7.5 mm, 15 mm, 17.5 mm, 22.5 mm, and/or 25 mm. In accordance with some forms the short protrusion distance comprises a protrusion distance having a length of about 10 mm and the large protrusion distance comprises a protrusion distance having a length of about 20 mm.

The methods described below begin with the device in a resting position, meaning that the depth stop member is in the extended position, the puller is fully inserted into the housing, and the slider is positioned at the distal end of the slider cavity. It will be understood that any method as described herein may begin with any suitable device configuration. In some forms, the disclosure provides for methods including placement of a guide cannula as detailed herein.

The present disclosure provides a method for obtaining a short biopsy sample. Such methods may include the steps of loading the device, firing the device, and/or removing a sample from the device. In certain embodiments, loading the device comprises retracting the puller (in a proximal direction) to a short sample position. At the short sample position the puller is partially retracted, causing the slider to move in a proximal direction and lock in place. The slider may be locked by any suitable means. For example, the slider may lock by passing a flexible arm over a locking surface of the housing, wherein once passed over the flexible arm extends and contacts the locking surface preventing movement in the proximal direction. In some forms, once locked in position the slider is biased towards the distal end of the housing by a firing spring which is put under tension by the movement of the slider. Thus, in some forms the device may be placed in a loaded position for a short sample collection, wherein the depth stop member is extended, the puller is partially retracted, and the slider is locked in position at a first locking surface. As detailed above, in some forms the stylet is attached to the puller and the cannula is attached to the slider. Thus, in the loaded position the distal end of the stylet is positioned at or near the distal end of the cannula. In accordance with some forms, the disclosed methods include inserting the biopsy needle into a previously placed guide cannula, such that the depth stop member contacts a proximal portion of the guide cannula, for example a hub assembly. In certain embodiments the device is configured such that in the loaded position when the depth stop member is in contact with the proximal end of the guide cannula, the distal end of the biopsy needle (e.g. the distal tip of the stylet and distal end of the cannula) are at or near the distal end of the guide cannula. In accordance with some forms, the step of firing the device comprises pushing the plunger (e.g. in a distal direction) causing the stylet to extend from the cannula a predetermined short distance into patient tissue, allowing patient tissue to fill the notch of the stylet. As the plunger is pushed further the slider is released from its locked position. For example, the slider may be released by a portion of the puller deflecting an arm of the slider pressing against a locking surface. Once released the firing spring pushes the slider to the distal end of the slider cavity, causing the cannula to extend from the guide cannula over the stylet, severing the tissue attached to the sample contained in the stylet notch. In accordance with some forms, the biopsy needle is removed from the guide cannula. To retrieve the sample the puller may be fully or partially retracted, locking the slider in a retracted state. In some forms the puller may be pushed in a distal direction without releasing this slider. In this way, the stylet is extended from the cannula and the sample may be retrieved.

The present disclosure provides a method for obtaining a long biopsy sample. Such methods may include the steps of loading the device, firing the device, and/or removing a sample from the device. In certain embodiments, loading the device comprises retracting the puller (in a proximal direction) to a long sample position. At the long sample position the puller is fully retracted, causing the slider to move in a proximal direction and lock in place. The slider may be locked by any suitable means. For example, the slider may lock by passing a flexible arm over a locking surface of the housing, wherein once passed over the flexible arm extends and contacts the locking surface preventing movement in the proximal direction. In some forms, once locked in position, the slider is biased towards the distal end of the housing by a firing spring put under tension by the slider's movement. In certain embodiments movement of the puller and/or slider to the long sample loaded position causes retraction of the depth stop member. For example, the depth stop member may be connected to an elongate control member positioned within the housing. In some forms, the control member comprises a proximal locking arm that when the device is configured in the depth stop extended position the proximal locking arm contacts a locking surface on the housing which prevents retraction of the depth stop member (in a proximal direction). In certain embodiments, the slider contacts and defects the proximal locking arm away from the locking surface allowing the depth stop member to be retracted. In some forms the slider may also contact a portion of the control member to cause movement of the control member, and depth stop member into the retracted position. The control member may comprise a distal locking arm configured to lock against a locking surface on the housing. In accordance with some forms, the distal locking arm engages the locking surface when the depth stop member is fully retracted and prevents movement of the depth stop member in the distal direction. Thus, in some forms the device may be placed in a loaded position for a long sample collection, wherein the depth stop member is retracted, the puller is fully retracted, and the slider is locked in position at a second locking surface. As detailed above, in some forms the stylet is attached to the puller and the cannula is attached to the slider. Thus, in the loaded position the distal end of the stylet is positioned at or near the distal end of the cannula. In accordance with some forms the disclosed methods include inserting the biopsy needle into a previously placed guide cannula, such that the distal surface of the housing, which may comprise the retracted depth stop member, contacts a proximal portion of the guide cannula, for example a hub assembly. In certain embodiments the device is configured such that in the loaded position when the distal end of the housing is in contact with the proximal end of the guide cannula, the distal end of the biopsy needle (e.g. the distal tip of the stylet and distal end of the cannula) are at or near the distal end of the guide cannula. In accordance with some forms, the step of firing the device comprises pushing the plunger (e.g. in a distal direction) causing the stylet to extend from the cannula a predetermined long distance into patient tissue, allowing patient tissue to fill the notch of the stylet. As the plunger is pushed further the slider is released from its locked position. For example, the slider may be released by a portion of the puller deflecting an arm of the slider pressing against a locking surface. Once released the firing spring pushes the slider to the distal end of the slider cavity, causing the cannula to extend from the guide cannula over the stylet, severing the tissue attached to the sample contained in the stylet notch. In accordance with some forms, the biopsy needle is removed from the guide cannula. To retrieve the sample the puller may be retracted, locking the slider in a retracted state. In some forms the puller may be pushed in a distal direction without releasing this slider. In this way, the stylet is extended from the cannula and the sample may be retrieved.

Devices of the present disclosure may be configured to reset the position of the depth stop member. Such a feature is advantageous, for example when sample sizes of various lengths are desired, or when removal of the sample results in retraction of the depth stop member. In certain embodiments, the device includes a depth stop mechanism spring configured to bias the depth stop member in a distal direction (e.g. to the extended configuration). In accordance with some forms, the device is configured to release the distal locking arm of the control member from its locked position allowing the depth stop mechanism spring to push the depth stop member to the extended configuration. In certain embodiments, as detailed above, the proximal locking arm on the control member locks against a locking surface of the housing to secure the depth stop member in the extended position.

Devices of the present disclosure may advantageously be manipulated with one hand. Such a configuration is particularly advantageous when a procedure must be carried out “in bore” when a patient is positioned within an MRI. With reference to FIG. 3A, the present disclosure provides a biopsy device having an angled plunger surface 1202 which transverses the longitudinal axis 1210 of the device at an angle extending from a proximal end 1204 positioned in line with the stylet notch to a distal end 1206, opposite the proximal end. In accordance with some forms, recessed portion 130 comprises proximal wall 1220 and distal wall 1212. In some forms, one or both of proximal wall 1220 and distal wall 1212 are angled at approximately the same angle as angled plunger surface 1202. In accordance with some forms, on each lateral side of the device, as shown in FIG. 3A, the recessed portion extends over the longitudinal axis of the device. In accordance with some forms, the recessed portion extends for at least two thirds of the surface of the housing on each of the lateral sides, preferably three quarters of the lateral surface from side to side.

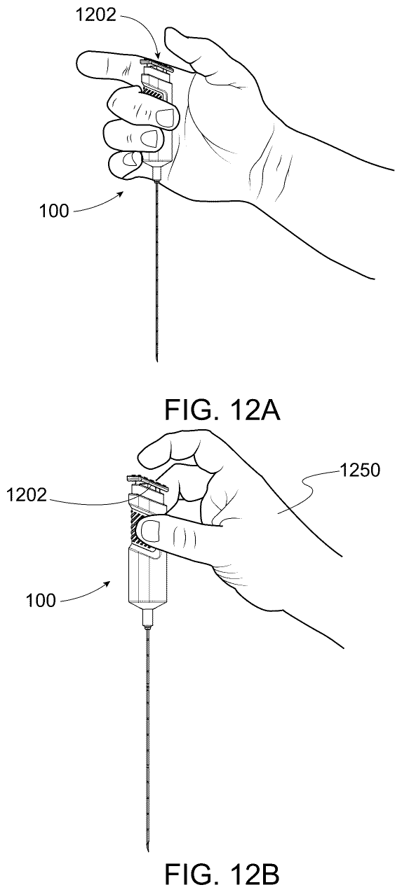

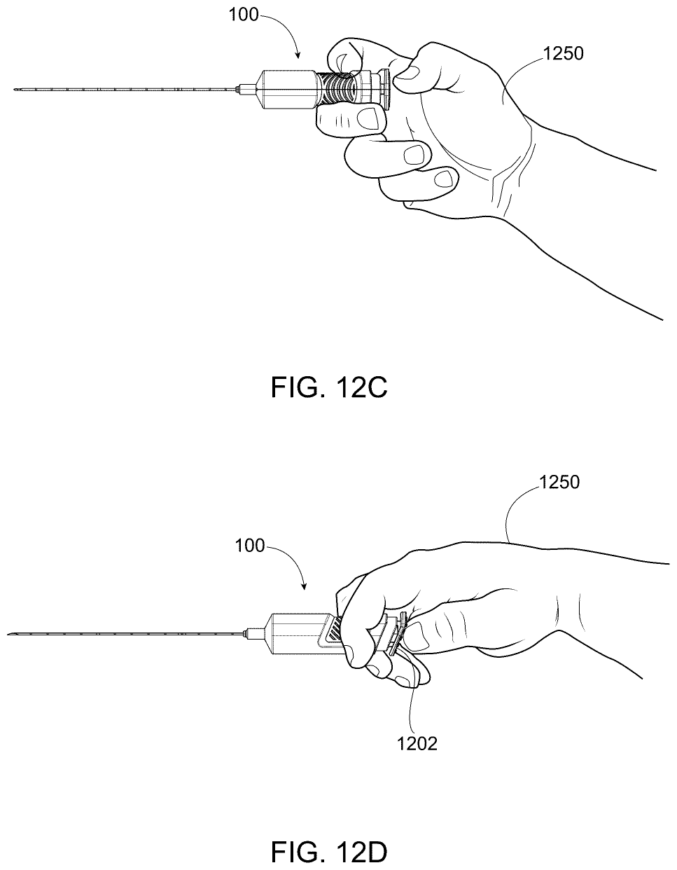

With reference to FIGS. 12A-12D, shown are alternative positions which the device may be held and operated by a user 1250 depending on whether the device is used for in-bore interventions or in-and-out interventions, as well as the nature of the procedure including, for example, changing the holding position depending on the organ being targeted. The holding position illustrated in FIG. 12A is suitable for in-bore interventions, and enables device activation with the thumb while the device is grasped with the index or middle finger of the user. The holding position illustrated in FIG. 12B is suitable for in-bore interventions, and enables longer triggering of the device inside the MRI bore. In this position the device is grasped between the thumb and middle or ring finger of the user and activated with the index finger. The holding positions illustrated in FIGS. 12C and 12D may be suitable for in-and-out interventions and allow for activation of the device with the user's thumb.

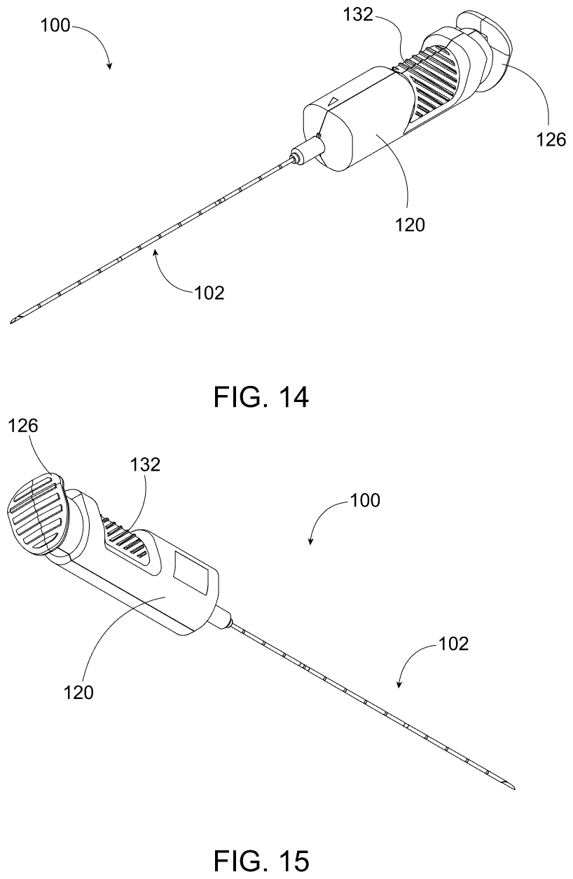

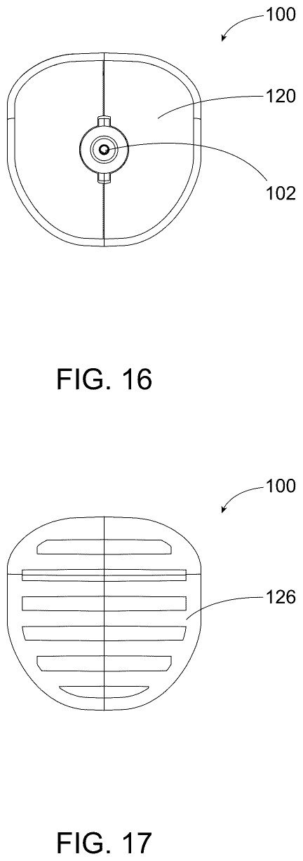

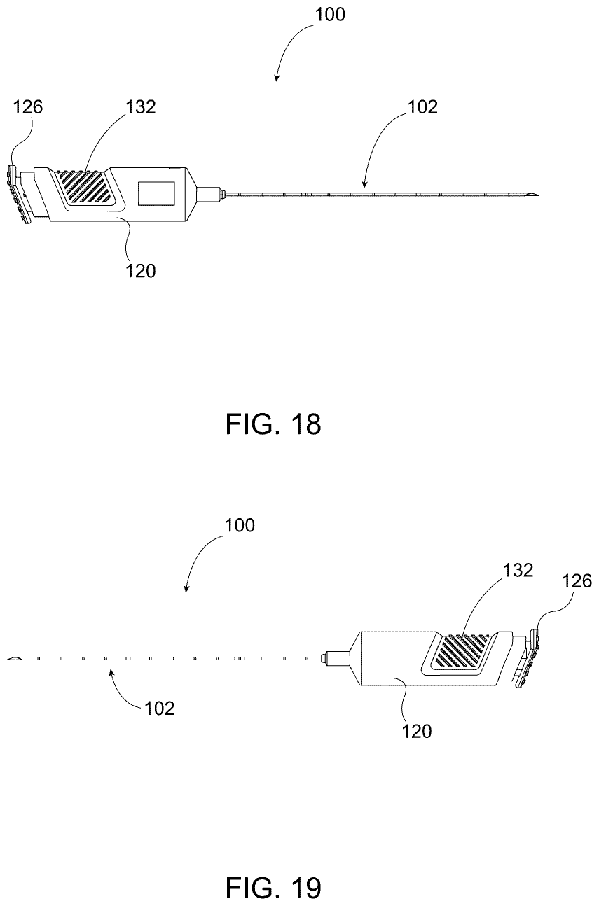

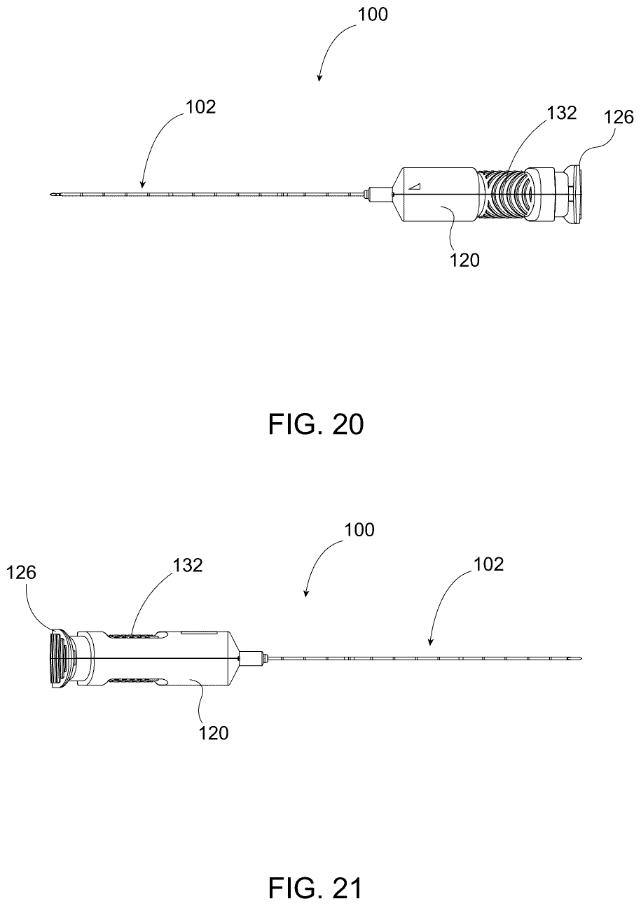

FIGS. 14-21 provide additional views of one embodiment of a biopsy device as provided herein.

The present disclosure also provides methods of using the biopsy devices disclosed herein. In some forms, such methods comprise inserting a biopsy needle through a guide cannula, the distal end of the guide cannula positioned near a target tissue. Wherein the biopsy device is configured to allow selection of the protrusion distance of the stylet from the biopsy needle cannula, and wherein the device is configured to adapt to either length selection such that the distal end of the biopsy needle is at the distal end of the guide cannula while a distal end of the biopsy device housing is in contact with the guide cannula hub. Such methods may also comprise firing the device. Such methods may include retracting the depth stop member to allow for a longer protrusion distance.

### EMBODIMENTS

The following provides an enumerated listing of some of the embodiments disclosed herein. It will be understood that this listing is non-limiting, and that individual features or combinations of features (e.g., 2, 3 or 4 features) as described in the Detailed Description above can be incorporated with the below-listed Embodiments to provide additional disclosed embodiments herein.
1. a Biopsy Device Comprising:
   a biopsy needle having a cannula and a stylet slidably received within the cannula;
   a housing having a distal end, wherein the biopsy needle extends from the distal end of the housing, the housing having a firing mechanism configured to extend the stylet relative to the cannula such that a distal end of the stylet protrudes from a distal end of the biopsy needle cannula by a selectable protrusion distance, wherein the selectable protrusion distance is adjustable between at least a first shorter distance and a second longer distance; and
   a depth stop member coaxially aligned around a portion of the biopsy needle, and wherein the depth stop member is configured to automatically adopt an extended configuration when the first protrusion distance is selected, and a retracted configuration when the second protrusion distance is selected.
2. The biopsy device of embodiment 1, comprising:
   a depth stop mechanism, the depth stop mechanism comprising an elongate control member contained within the housing, and the depth stop member positioned at a distal end of the control member.
3. The biopsy device of embodiment 2, wherein the elongate control member comprises one or more deflectable arms configured to lock the depth stop member into the extended configuration and/or the retracted configuration.
4. The biopsy device of any one of the preceding embodiments, wherein the stylet comprises a sampling notch positioned near the stylet distal end.
5. The biopsy device of embodiment 4, wherein the stylet comprises:

one or more MRI visible markers positioned near the sampling notch.
6. The biopsy device of any one of embodiments 4 or 5, wherein the housing comprises a notch indicia axially aligned with the sampling notch.
7. The biopsy device of any one of the preceding embodiments, wherein the biopsy needle cannula comprises a cutting edge positioned at the distal end.
8. The biopsy device of any one of the preceding embodiments, wherein in the extended configuration the depth stop member extends from the housing by a length about the difference between the first protrusion distance and the second protrusion distance.
9. The biopsy device of any one of the preceding embodiments, wherein the first protrusion distance is about 10 mm.
10. The biopsy device of any one of the preceding embodiments, wherein the second protrusion distance is about 20 mm.
11. The biopsy device of any one of the preceding embodiments, wherein the depth stop member comprises a distal end configured to engage a guide cannula hub.
12. The biopsy device of any one of the preceding embodiments, wherein the firing mechanism comprises a puller defining a slider cavity, and a slider positioned within the slider cavity.
13. The biopsy device of embodiment 12, wherein the puller further comprises a plunger positioned at a proximal end of the puller.
14. The biopsy device of embodiment 13, wherein the plunger comprises an angled plunger surface.
15. The biopsy device of any one of embodiments 12 to 14, wherein the stylet is configured to move with the puller.
16. The biopsy device of any one of embodiments 12 to 15, wherein the firing mechanism comprises a slider positioned within the slider cavity.
17. The biopsy device of embodiment 16, wherein the biopsy needle cannula is configured to move with the slider.
18. The biopsy device of any one of embodiments 16 or 17, comprising:
   a firing spring configured to bias the slider towards a distal end of the slider cavity.
19. The biopsy device of any one of embodiments 1 to 18, comprising:
   a depth stop mechanism spring configured to bias the depth stop mechanism in a distal direction.
20. The biopsy device of any one of embodiments 16 to 19 as dependent on claims 2 or 3, wherein the slider is configured to release a proximal locking arm of the depth stop mechanism releasing the depth stop mechanism from the extended position.
21. A biopsy kit, comprising:
   a biopsy device of any one of embodiments 1 through 20; and
   a coaxial guide needle sized to receive the biopsy needle, and configured such that upon insertion of the biopsy needle:
      wherein the depth stop member is configured to contact a proximal end of the coaxial guide needle when the first protrusion distance is selected, such that the distal end of the biopsy needle aligns with the distal end of the coaxial guide needle; and
      the distal end of the housing is configured to contact a proximal end of the coaxial guide needle, such that a distal end of the coaxial guide needle aligns with a distal end of the biopsy needle when the first protrusion distance is selected.
22. A method of obtaining a tissue sample using the device of any one of embodiments 1-20, the method comprising:
   inserting the biopsy needle through a guide cannula, the distal end of the guide cannula positioned near a target tissue, wherein a distal portion of the housing contacts a hub body of the guide cannula when the distal end of the biopsy needle is positioned at the distal end of the guide cannula.
23. The method of embodiment 22, comprising: retracting the depth stop member into the housing.

It is intended that the foregoing detailed description be regarded as illustrative rather than limiting, and that it be understood that it is the following claims, including all equivalents, that are intended to define the spirit and scope of this invention. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

1. A biopsy device comprising:
a biopsy needle having a cannula and a stylet slidably received within the cannula;
a housing having a distal end, wherein the biopsy needle extends from the distal end of the housing, the housing having a firing mechanism configured to extend the stylet relative to the cannula such that a distal end of the stylet protrudes from a distal end of the biopsy needle cannula by a selectable protrusion distance, wherein the selectable protrusion distance is adjustable between at least a first shorter distance and a second longer distance; and
a depth stop member coaxially aligned around a portion of the biopsy needle, and wherein the depth stop member is configured to automatically adopt an extended configuration when the first protrusion distance is selected, and a retracted configuration when the second protrusion distance is selected.

2. The biopsy device of claim 1, comprising:
a depth stop mechanism, the depth stop mechanism comprising an elongate control member contained within the housing, and the depth stop member positioned at a distal end of the control member.

3. The biopsy device of claim 2, wherein the elongate control member comprises one or more deflectable arms configured to lock the depth stop member into the extended configuration and/or the retracted configuration.

4. The biopsy device of claim 1 wherein the stylet comprises a sampling notch positioned near the stylet distal end.

5. The biopsy device of claim 4, wherein the stylet comprises:
one or more MRI visible markers positioned near the sampling notch.

6. The biopsy device of claim 4, wherein the housing comprises a notch indicia axially aligned with the sampling notch.

7. The biopsy device of claim 1, wherein the biopsy needle cannula comprises a cutting edge positioned at the distal end.

8. The biopsy device of claim 1, wherein in the extended configuration the depth stop member extends from the housing by a length about the difference between the first protrusion distance and the second protrusion distance.

9. The biopsy device of claim 1, wherein the first protrusion distance is about 10 mm.

10. The biopsy device of claim 1, wherein the second protrusion distance is about 20 mm.

11. The biopsy device of claim 1, wherein the depth stop member comprises a distal end configured to engage a guide cannula hub.

12. The biopsy device of claim, wherein the firing mechanism comprises a puller defining a slider cavity, and a slider positioned within the slider cavity.

13. The biopsy device of claim 12, wherein the puller further comprises a plunger positioned at a proximal end of the puller.

14. The biopsy device of claim 13, wherein the plunger comprises an angled plunger surface.

15. The biopsy device of claim 12, wherein the stylet is configured to move with the puller.

16. The biopsy device of claim 12, wherein the firing mechanism comprises a slider positioned within the slider cavity.

17. The biopsy device of claim 16, wherein the biopsy needle cannula is configured to move with the slider.

18. The biopsy device of claim 16, comprising:
a firing spring configured to bias the slider towards a distal end of the slider cavity.

19. The biopsy device of claim 1, comprising:
a depth stop mechanism spring configured to bias the depth stop mechanism in a distal direction.

20. The biopsy device of claim 16 as dependent on claim 2, wherein the slider is configured to release a proximal locking arm of the depth stop mechanism releasing the depth stop mechanism from the extended position.

21. A biopsy kit, comprising:
a biopsy device of claim 1; and
a coaxial guide needle sized to receive the biopsy needle, and configured such that upon insertion of the biopsy needle:
wherein the depth stop member is configured to contact a proximal end of the coaxial guide needle when the first protrusion distance is selected, such that the distal end of the biopsy needle aligns with the distal end of the coaxial guide needle; and
the distal end of the housing is configured to contact a proximal end of the coaxial guide needle, such that a distal end of the coaxial guide needle aligns with a distal end of the biopsy needle when the first protrusion distance is selected.

22. A method of obtaining a tissue sample using the device of claim 1, the method comprising:
inserting the biopsy needle through a guide cannula, the distal end of the guide cannula positioned near a target tissue, wherein a distal portion of the housing contacts a hub body of the guide cannula when the distal end of the biopsy needle is positioned at the distal end of the guide cannula.

23. The method of claim 22, comprising:
retracting the depth stop member into the housing.
